(19) United States
(12) Patent Application Publication
(10) Pub. No.: US 2025/0249229 A1
(43) Pub. Date: Aug. 7, 2025

(54) EXPANDABLE MECHANICAL HEMODYNAMIC SUPPORT SYSTEMS, DEVICES, AND METHODS

(71) Applicants: Narwhal Medical, Inc., Minneapolis, MN (US); Boston Scientific Scimed, Inc., Maple Grove, MN (US)

(72) Inventors: Loïc Van Horne, Minneapolis, MN (US); Qian Liu, Plymouth, MN (US); Aaron Chalekian, Savage, MN (US)

(21) Appl. No.: 19/045,223

(22) Filed: Feb. 4, 2025

Related U.S. Application Data
(60) Provisional application No. 63/549,750, filed on Feb. 5, 2024.

(51) Int. Cl. A61M 60/17; A61M 60/13; A61M 60/216; A61M 60/414; A61M 60/808; A61M 60/81; A61M 60/90

(52) U.S. Cl. CPC A61M 60/17; A61M 60/13; A61M 60/216; A61M 60/414; A61M 60/808; A61M 60/81; A61M 60/90; A61M 2205/04; A61M 2205/103; A61M 2210/125; A61M 2210/127

(57) ABSTRACT

Blood pump systems such as percutaneous ventricular assist devices (“pVADs”) can be used to provide hemodynamic support to patients. Some embodiments of pVADS include an inlet cannula and a pump housing. The inlet cannulae and pump housings of example pVADs may be reconfigurable between a low-profile delivery configuration and a radially expanded operable configuration. In some embodiments, a pusher rod is used to releasably apply axial tension to the inlet cannulae and pump housings to thereby configure those components in the low-profile delivery configuration.

### CROSS-REFERENCE TO RELATED APPLICATIONS

This application claims the benefit of U.S. Provisional Application Ser. No. 63/549,750 filed Feb. 5, 2024. The disclosure of the prior application is considered part of (and is incorporated by reference in) the disclosure of this application.

### TECHNICAL FIELD

This disclosure relates to mechanical hemodynamic support systems, devices, and methods, such as expandable percutaneous ventricular assist devices and usage methods.

### BACKGROUND

Mechanical hemodynamic support devices, such as percutaneous ventricular assist devices (“pVADs”) and other devices, are currently used in the practice of interventional cardiology to perform protected percutaneous coronary intervention (“PCI”). During such procedures, mechanical hemodynamic support is either placed prophylactically or readily available in the event of a complication. If a complication occurs, having hemodynamic support to continue circulating blood throughout the body while the complication is mitigated provides significant patient benefits.

Additionally, pVADs can be used to offload the heart prior to performing PCI. As a result, the work required by the heart to pump blood is reduced because the pVAD takes on a significant portion of the pumping load. There is clinical evidence showing that offloading the heart during a myocardial infarction has long-term beneficial effects for myocardial tissue viability.

Current pVADs require a relatively large access site to accommodate delivery. Large access site requirements require vascular access via large vessels such as the femoral artery, the axillary artery, or in the venous system. Large access sites require longer patient follow-up and are more prone to bleeding complications than smaller access sites.

Additionally, the sizes of current pVADs are too large for many patients throughout the world, such as children and people with smaller body structures. Accordingly, improved systems, devices, and methods would be beneficial.

### SUMMARY

This disclosure describes blood pump systems such as pVADs, and methods for their deployment and use. This disclosure is further focused specifically on the blood cannula and pump housing components of pVADs.

The pump housings of the example pVADs described herein may be reconfigurable between a low-profile delivery configuration and a radially expanded operable configuration. The pVADs may also comprise an inlet cannula extending from the pump housing. In some embodiments, the inlet cannula is reconfigurable between a low-profile delivery configuration and a radially expanded operable configuration. This disclosure also describes example pVADs that include an elongate delivery sheath device defining a sheath lumen, a housing component slidably disposable in the sheath lumen, and an internal pusher rod used for axially tensioning and thereby collapsing the expandable pump housing and/or inlet cannula. The housing component includes an elongate drive shaft housing defining a housing lumen, a pump housing disposed at a distal end portion of the drive shaft housing, and an inlet cannula extending from the pump housing. The pump housing and the inlet cannula are radially reconfigurable between: (i) low-profile delivery configurations when radially collapsed and (ii) radially expanded operable configurations when allowed to conform to predetermined biased geometry.

This disclosure also describes methods for deploying and operating blood pump systems such as pVADs. In one example, such a method for deploying a pVAD to a target location within a patient includes advancing, through an incision of the patient, an elongate delivery sheath device. The delivery sheath device may define a sheath lumen that contains a housing component slidably disposed in the sheath lumen. Such a housing component may include an elongate drive shaft housing defining a housing lumen, a pump housing disposed at a distal end portion of the drive shaft housing, and an inlet cannula extending from the pump housing. The method may also include retracting, when the pump housing is at the target location, the internal pusher rod and/or the delivery sheath device relative to the housing component to express the inlet cannula and the pump housing out from the sheath lumen. The pump housing and the inlet cannula may be radially reconfigurable between: (i) low-profile delivery configurations when radially constrained and (ii) radially expanded operable configurations when unconstrained by the delivery sheath and/or internal pusher rod.

Such a method for deploying a percutaneous blood flow assist device to a target location within a patient may optionally include one or more of at least the following features. The pVADs may be percutaneously deployed through an incision that provides access to a femoral artery of the patient, and the delivery sheath device may be advanced to the target location via the femoral artery. Alternately, the incision may provide access to a radial artery of the patient, and the delivery sheath device may be advanced to the target location via the radial artery. In other cases, the incision may provide access to a thoracic cavity of the patient, and the delivery sheath device may be advanced to the target location via the thoracic cavity.

The target location at which the pVADs are deployed may be an aortic valve region of the patient, including the left ventricle and aorta, for example.

In one aspect, this disclosure is directed to a percutaneous ventricular assist blood pump system. Such a pump system can include a pump housing component comprising: (i) an elongate drive shaft housing; (ii) a hub attached at a proximal end of the drive shaft housing; (iii) a pump housing attached at a distal end of the drive shaft housing; (iv) an inlet cannula extending distally from the pump housing; and (v) a tip portion extending distally from the inlet cannula. Such a pump system may also include a pusher rod comprising a shaft and a hub. The blood pump system is reconfigurable between a low-profile delivery configuration and a radially expanded operable configuration. While the percutaneous ventricular assist blood pump system is configured in the low-profile delivery configuration: (1) the hub of the pusher rod is releasably coupled to the hub of the pump housing component; (2) the shaft of the pusher rod extends through: (i) the hub of the pump housing component, (ii) the elongate drive shaft housing, (iii) the pump housing, and (iv) the inlet cannula; and (3) a distal tip portion of the shaft of the pusher rod exerts distally directed force on the tip portion of the pump housing to create axial tension on: (a) the elongate drive shaft housing, (b) the pump housing, and (c) the inlet cannula. The axial tension causes radial contraction of the pump housing and the inlet cannula. The pump housing and the inlet cannula are radially expandable to the operable configuration when the axial tension is eliminated by uncoupling the hub of the pusher rod from the hub of the pump housing component and pulling the pusher rod proximally in relation to the pump housing component.

Such a percutaneous ventricular assist blood pump system may optionally include one or more of the following features. The system may also include an introducer sheath slidably disposed over the pump housing and inlet cannula while the percutaneous ventricular assist blood pump system is configured in the low-profile delivery configuration. The tip portion of the pump housing component may include a tapered outer diameter that is configured to dilate a vascular access site to allow the rest of the percutaneous ventricular assist blood pump system to enter through the vascular access site. In some embodiments, in the operative configuration, the tip portion of the pump housing component is J-shaped. In some embodiments, in the operative configuration, the tip portion of the pump housing component is shaped like a question mark with two curves in opposite directions. A proximal-most curve of the two curves may be sufficiently rigid to bias a direction of a guidewire within a central lumen of the tip portion. A distal-most curve of the two curves may be pliable enough to conform to a linear direction of the guidewire when a guidewire is present within the central lumen. The distal-most curve may reconfigure to an atraumatic geometry when the guidewire is removed from the central lumen. In some embodiments, the tip portion of the pump housing against which the distal tip portion of the shaft of the pusher rod exerts the distally directed force comprises an annular flat surface that is flush with an expandable structure of the inlet cannula. The tip portion of the pump housing against which the distal tip portion of the shaft of the pusher rod may exert the distally directed force comprises an annular flat surface that is within an expandable structure of the inlet cannula. The tip portion of the pump housing against which the distal tip portion of the shaft of the pusher rod may exert the distally directed force comprises a proximal face with a concave chamfer. The tip portion of the pump housing against which the distal tip portion of the shaft of the pusher rod may exert the distally directed force comprises a generally spherical proximal end portion. The tip portion of the pump housing against which the distal tip portion of the shaft of the pusher rod may exert the distally directed force comprises a cap member.

In another aspect, this disclosure is directed to a percutaneous ventricular assist blood pump system that includes a pump housing component comprising: (i) an elongate drive shaft housing; (ii) a radially-expandable pump housing attached at a distal end of the drive shaft housing; and (iii) a radially-expandable inlet cannula extending from the pump housing. The percutaneous ventricular assist blood pump system is reconfigurable between a low-profile delivery configuration and a radially expanded operable configuration. While the percutaneous ventricular assist blood pump system is configured in the low-profile delivery configuration, the pump housing and inlet cannula are in a state of axial tension. While the percutaneous ventricular assist blood pump system is configured in the radially expanded operable configuration, the pump housing and inlet cannula are relieved of the axial tension.

In another aspect, this disclosure is directed to a method of deploying a percutaneous ventricular assist blood pump system. The method includes: advancing a pump housing component to a heart of a patient while the pump housing component is in a low-profile delivery configuration resulting from tensioning the pump housing component using a pusher rod that is releasably coupled with the pump housing component; and uncoupling the pusher rod from the pump housing component to relieve the tensioning and to allow the pump housing component to self-expand to an operable configuration. In some embodiments, the method also includes advancing an inner catheter comprising a pump impeller and flexible drive shaft into the pump housing component to assemble the percutaneous ventricular assist blood pump system in vivo.

In another aspect, this disclosure is directed to a medical device system that includes: (i) a radially expandable medical device; and (ii) a pusher rod that is releasably coupleable with the radially expandable medical device. The radially expandable medical device is held in a low-profile delivery configuration while the pusher rod is coupled with the radially expandable medical device to apply longitudinal tension to the radially expandable medical device. The radially expandable medical device is configured to self-expand to an operable configuration when the pusher rod is uncoupled from the radially expandable medical device to relieve the longitudinal tension from the radially expandable medical device.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTIONS OF THE DRAWINGS

FIG. 1 illustrates an example mechanical hemodynamic support device (or pVAD) positioned in the heart of a patient to pump blood from the left ventricle into the aorta, in accordance with embodiments of the subject matter disclosed herein.

FIGS. 2A-2D illustrate an example collapsible mechanism that can be used for delivery of a pump housing and cannula, in accordance with embodiments of the subject matter disclosed herein.

FIGS. 3A and 3B illustrate exemplary distal end component interactions of a collapsible mechanism of a pump housing and cannula, in accordance with embodiments of the subject matter disclosed herein.

FIGS. 4A-4E illustrate example embodiments of internal distal tip components of a collapsible pump housing and cannula, in accordance with embodiments of the subject matter disclosed herein.

FIGS. 5A-5D illustrate sequential steps of an example delivery process of a collapsible pump housing and cannula, in accordance with embodiments of the subject matter disclosed herein.

FIGS. 6A-6E illustrate sequential steps of another example delivery process of a collapsible pump housing and cannula, in accordance with embodiments of the subject matter disclosed herein.

FIGS. 7A and 7B illustrate an example distal tip used for delivery of a collapsible pump housing and cannula, in accordance with embodiments of the subject matter disclosed herein.

FIG. 8 illustrates another example mechanical hemodynamic support device (or pVAD) positioned in the heart of a patient to pump blood from the left ventricle into the aorta, in accordance with embodiments of the subject matter disclosed herein.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Mechanical hemodynamic support devices, such as pVADs according to embodiments of the present disclosure, are capable of unloading or assisting the heart of a patient for a period of time during, for example, a myocardial infarction, cardiogenic shock, a surgical or interventional procedure, and the like.

In some embodiments, the pVAD devices described herein have a sufficiently small profile to facilitate deployment to the heart of a patient through a radial artery access site. Some such embodiments of the pVAD devices described herein have an innovative two-part design that allows very small delivery profiles (as required by the radial artery access site, for example). The pVAD devices described herein can also be deployed via other vascular access sites such as, but not limited to, the femoral artery, the axillary artery, and the venous system, to provide a few non-limiting examples.

After the device has been advanced to a desired location, or a target location, for example within the heart of a patient, it is radially expanded from its deployment profile (also referred to as a low-profile or collapsed configuration), to its functional pumping size (also referred to as an expanded or operable configuration). PVADs according to embodiments of the present disclosure can be configured to provide various flow outputs.

This disclosure describes example design and delivery techniques of the pump housing component of pVADs, which may be the first component delivered of the two-part delivery of the pVADs described herein.

As used herein, the terms “proximal” and “distal” pertain to the orientation of the devices, not the patient. For example, proximal portions of the devices described herein may reside outside of the patient for manipulation by a clinician operator, while distal portions of the devices are residing within the patient.

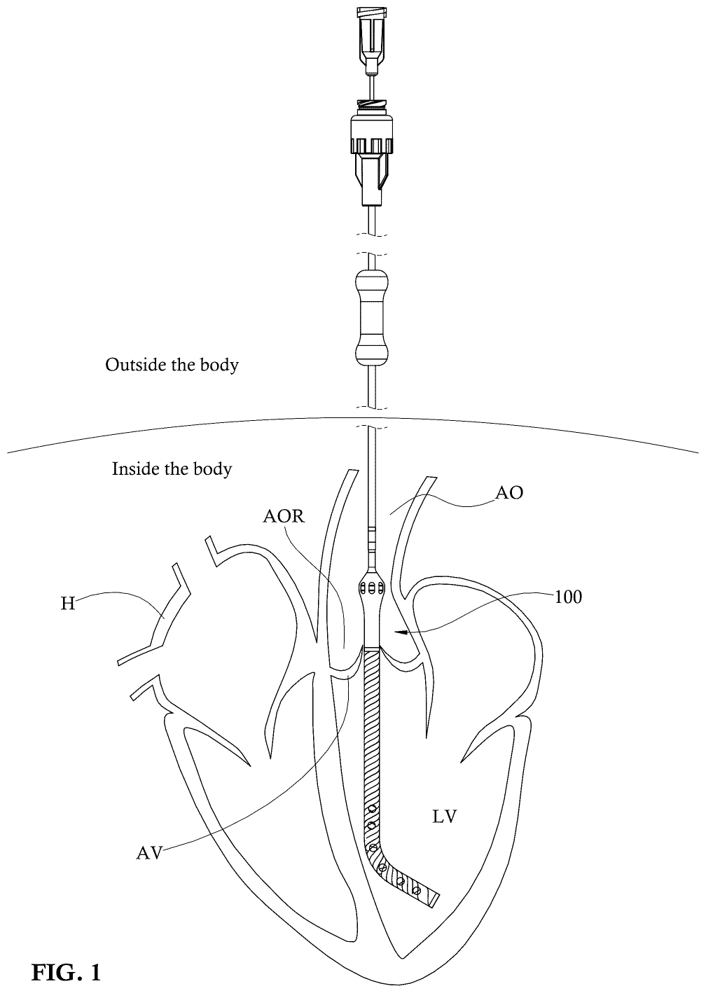

FIG. 1 illustrates an example mechanical hemodynamic support device 100 (which may also be referred to as “pVAD 100” or “pump device 100”) positioned in the heart H of a patient, in accordance with some embodiments of the subject matter disclosed herein. In the illustrated arrangement, the pump device 100 reaches the heart H by advancing it via a vascular access site and through the vasculature of the patient while a pump housing component of the pump device 100 is maintained in a low-profile delivery configuration, as described further below. In the depicted example, once the device is in the desired anatomical location, the pump housing component is expanded to its operating size. Then, a separate inner catheter comprising an expandable pump impeller (e.g., an inflatable pump impeller) and a flexible drive shaft is advanced by the clinician into the pump housing component to construct or assemble the two-part pump device 100 in vivo. The pump impeller can be expanded (e.g., inflated) after it has been positioned in the pump housing. Various embodiments and uses of such a pump device 100 are disclosed, for example, in U.S. patent application Ser. No. 18/108,409 (now U.S. Pat. No. 12,090,313) which is hereby incorporated by reference in its entirety and for all purposes.

Still referring to FIG. 1, in the depicted example embodiment the distal tip portion of the pump device 100 is located in the left ventricle (LV). The pump device 100 draws blood out of the ventricle LV by the pump impeller in the pump housing and then delivers the blood at a higher pressure to the aortic root (“AOR”) via one or more blood outlets of the pump housing positioned in the AOR. The blood is then circulated throughout the body of the patient by the vasculature of the patient. The pump may also be configured to be placed in any other anatomical location where circulatory support may be needed. Non-limiting examples may include placing the pump in the right ventricle, in the descending aorta near the renal arteries, in the carotid arteries, or at the iliac arch.

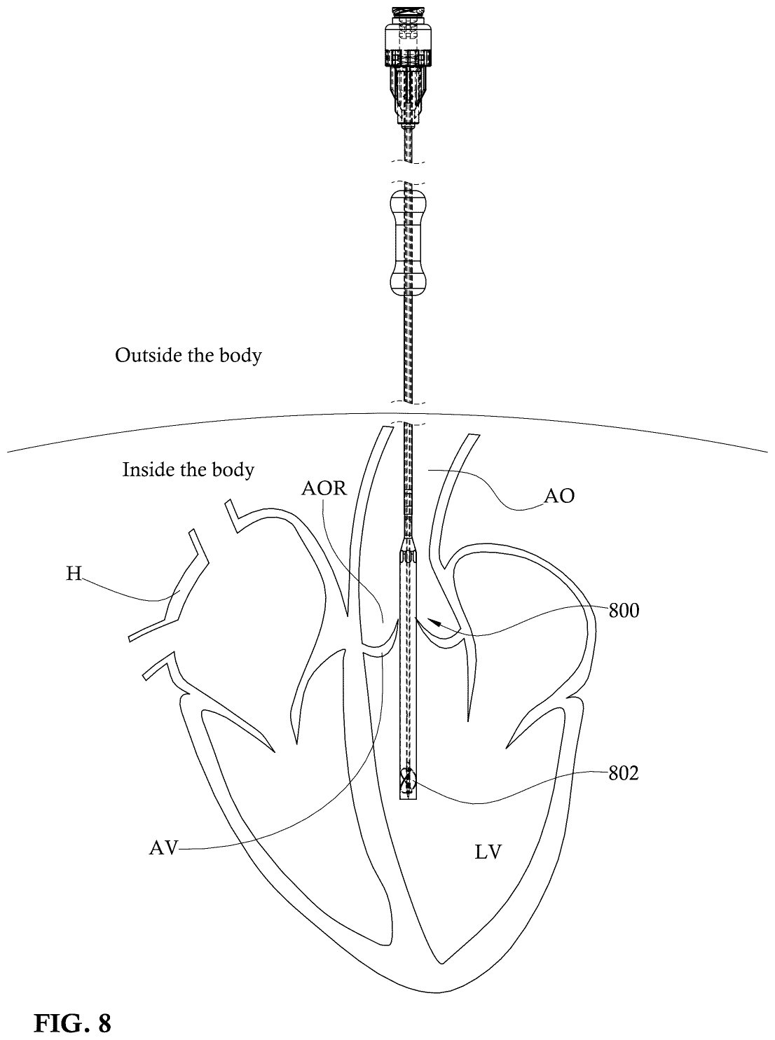

Referring also to FIG. 8, in some embodiments, such as the pVAD 800, the expandable impeller 802 can positioned in the LV at a distal end portion of the expandable cannula and pump housing, rather than in the pump housing located in the AOR as in the example pump device 100 of FIG. 1. The pVAD 800 can be deployed into the heart H using the techniques described herein.

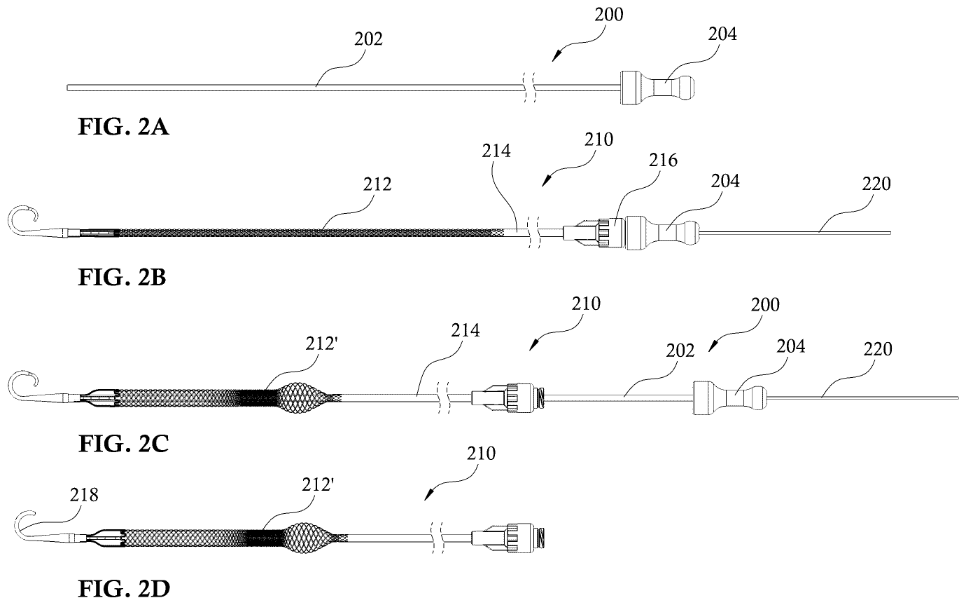

FIGS. 2A-2D illustrate an example pVAD embodiment that includes components for maintaining a pump housing in a radially collapsed delivery/retrieval configuration while tracking the pump housing component through a patient's vasculature. In this embodiment, the pump housing and inlet cannula has a natural bias toward being in the expanded configuration. Some non-limiting examples of materials that can be used to create the self-expanding bias include shape memory alloys like Nitinol that can allow a scaffold to be collapsed for delivery and to self-expand to an operable configuration when tension is removed from the scaffold. The shape memory scaffold may be surrounded by an elastomeric membrane made of materials, such as but not limited to, a silicone or polyurethane, thereby allowing the membrane to conform to the shape of the scaffold while simultaneously causing the walls of the inlet cannula and pump housing to be impermeable. In some embodiments, the blood inlet and blood outlet may have portions of the scaffold not covered in an elastomeric membrane.

FIG. 2A illustrates a pusher rod 200 which has two separate components, i.e., a pusher rod shaft 202 and an optional pusher rod hub 204. The pusher rod shaft 202 may be designed to have sufficient column strength to exert axial/longitudinal compression against the distal tip portion of the pump housing while also being laterally flexible enough to be delivered though the necessary vasculature. The pusher rod shaft 202 may be made of materials such as, but not limited to, PTFE, LDPE, PEEK, a nylon-based polymer, or a laser cut metallic hypotube. Additionally, in some embodiments the pusher rod shaft 202 may be coated to reduce the friction between the pusher rod shaft 202 and the drive shaft housing 214 while navigating through the vasculature.

FIG. 2B illustrates the pump housing system 210 in its collapsed low-profile delivery configuration. The collapsed pump housing and inlet cannula 212 is attached to a distal end of the drive shaft housing 214, which in turn is connected to the drive shaft housing hub 216. In the depicted embodiment, the drive shaft housing hub 216 is releasably coupled to the pusher rod hub 204, and the pusher rod shaft (not shown) is inside the pump housing system 210 and internally pushing against the distal tip 218 of the pump housing and inlet cannula 212, thereby creating tension along the pump housing and inlet cannula 212 to maintain the pump housing and inlet cannula 212 in the collapsed low-profile delivery configuration. The pump housing system 210 in its collapsed configuration may be tracked over a guidewire 220 to the desired anatomical location within a patient. The distal tip 218 of the pump housing and inlet cannula 212 as well as the pusher rod 200 may have a central lumen to allow the guidewire 220 to be tracked concentrically within the pump housing system 210 in its collapsed low-profile delivery configuration as shown in FIG. 2B.

FIG. 2C illustrates the expansion of the pump housing and inlet cannula 212′ by the retraction of the pusher rod 200 from inside the pump housing system 210. When the pusher rod 200 is retracted, it removes tension that was previously exerted by the pusher rod 200 along the pump housing and inlet cannula 212′, thereby allowing pump housing and inlet cannula 212′ to return to its natural, biased expanded state. In the expanded state, the inlet cannula 212′ may have, but is not limited to, an outer diameter in the range of 4 mm to 8 mm. The maximum diameter of the pump housing in the expanded state may have, but is not limited to, an outer diameter in the range of 6 mm to 12 mm. The pump housing and inlet cannula 212′ in the expanded state may have, but is not limited to, a length in the range of 5 cm to 15 cm.

In the depicted example embodiment, the retraction of the pusher rod 200 is made possible by first de-coupling the pusher rod hub 204 from the drive shaft housing hub 216. However, such decoupling of the pusher rod 200 from the pump housing system 210 is not required in all cases. In some cases, the pusher rod 200 can be exerting tension on the pump housing system 210 without the pusher rod 200 being coupled to the pump housing system 210. For example, in some cases a clinician can manually maintain the pusher rod 200 in a position relative to the pump housing system 210 so that the pusher rod 200 exerts force on the pump housing system 210 to keep the pump housing system 210 in tension, and thereby in its low-profile delivery configuration.

FIG. 2D illustrates the pump housing system 210 with the pump housing and inlet cannula 212′ in its expanded state with the pusher rod 200 and the guidewire 220 fully removed from the radially expanded pump housing and inlet cannula 212′.

The pump housing and inlet cannula 212′ can be radially re-collapsed down by re-inserting the pusher rod 200 into the system and re-applying the axial tensile force across the pump housing and inlet cannula 212, when needed.

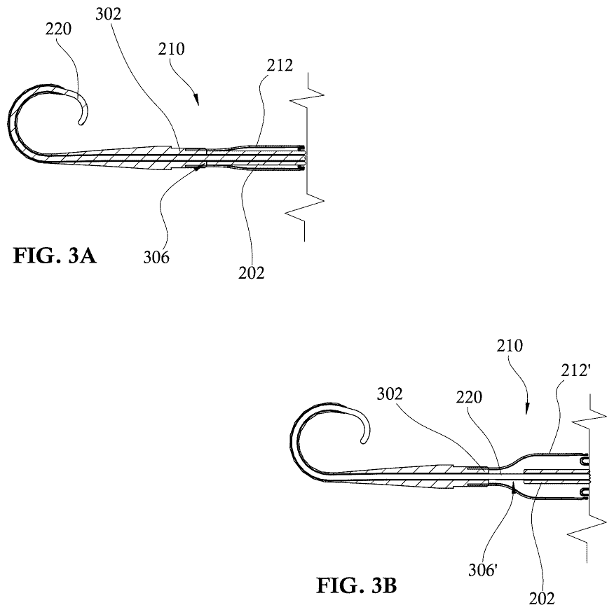

FIG. 3A illustrates a longitudinal cross-sectional view of the distal tip portion of the pump housing system 210 while the distal tip of the pusher rod shaft 202 is exerting distally directed force on the proximal end surface 302 of the distal end member of the pump housing system 210. The distally directed force, in combination with the coupling of the drive shaft housing hub 216 from the pusher rod hub 204 (see FIG. 2B), puts the pump housing and inlet cannula 212 in tension so that the diameter of the pump housing and inlet cannula 212 are in their low-profile delivery configurations as shown in FIGS. 2B and 3A. Some non-limiting examples of the delivery profile for the pump housing and inlet cannula 212 when they are in their low-profile delivery configuration may be for them to be compatible with introducer sheaths in the range of 5 French to 12 French. Therefore, the maximum diameter of the distal tip 218 may be less than the internal diameter of the introducer sheath being used. Alternatively, the maximum diameter of the distal tip 218 may be equivalent to the outer diameter of the introducer sheath being used, therefore allowing the distal tip 218 to act as a dilator for introducing the introducer sheath into the vasculature.

FIG. 3B illustrates a gap 306′ between the proximal end surface 302 of the distal end member of the pump housing system 210 and the distal tip of the pusher rod shaft 202. This gap 306′ can be created, for example, by uncoupling the drive shaft housing hub 216 from the pusher rod hub 204 (see FIG. 2C) and then pulling the pusher rod shaft 202 proximally in relation to the distal end member of the pump housing system 210. This may be done while simultaneously removing the guidewire 220, or alternatively may be performed while leaving the guidewire 220 in place within the system. Those actions also remove the tensile force from the pump housing and inlet cannula 212′ so that the pump housing and inlet cannula 212′ are free to reconfigure (e.g., radially expand and longitudinally shorten) to their natural radially expanded configuration as depicted in FIG. 3B.

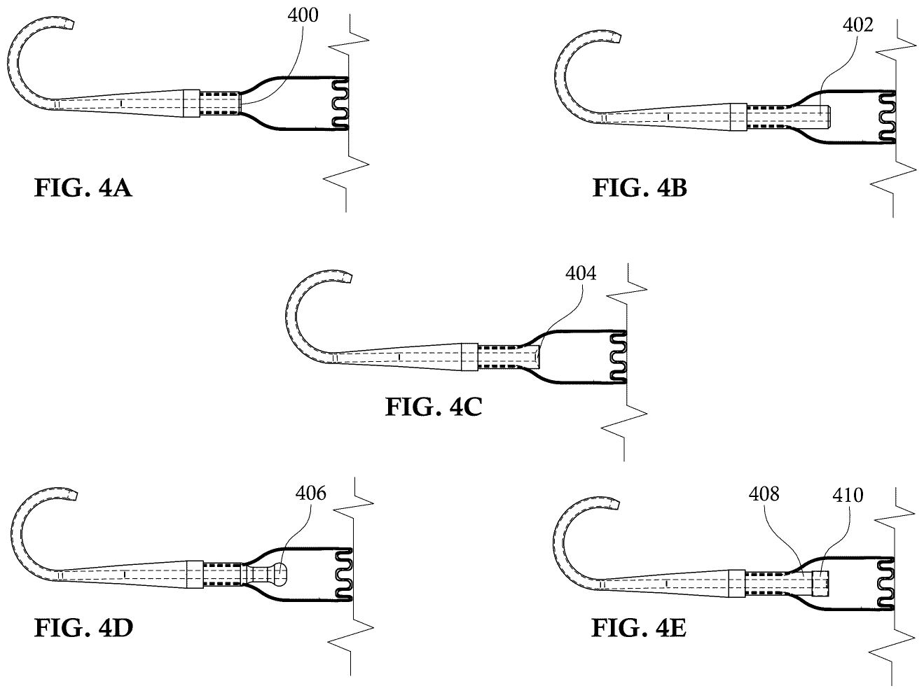

FIGS. 4A-4E illustrate various alternative embodiments of the proximal end surface 302 (shown elsewhere) of the distal end member of the pump housing and inlet cannula 212. The proximal end surface 302 of the distal end member of the pump housing and inlet cannula 212 may be designed to allow for the distal end of the pusher rod shaft 202 to butt up against it (e.g., engage against, exert force against, press against, releasably couple with, etc.) but to not get stuck to it when pressure is applied. For example, FIG. 4A shows an annular flat surface 400 which is flush with the expandable structure of the inlet cannula. FIG. 4B shows a protruding proximal end surface 402 which may decrease the chances for the pusher rod to get wedged into the expandable structure. FIG. 4C illustrates a proximal end surface 404 with a concave chamfer which may facilitate alignment between proximal edge 404 and the distal end of the pusher rod shaft 202. FIG. 4D illustrates another example embodiment, in which the proximal end surface 406 is generally spherical. FIG. 4E illustrates a protruding proximal end surface with a cap 410 placed over the end. The cap 410 may function as a shield to increase the robustness of the proximal end of the distal end member of the pump housing an inlet cannula 212, which could be beneficial if the protruding proximal member 408 is a polymer which may soften during the course of device implantation. These examples are illustrative in nature and are not to be construed as limiting, still other configurations or combinations of these features will be apparent to those experienced in the field, and the intent is to cover those embodiments as well.

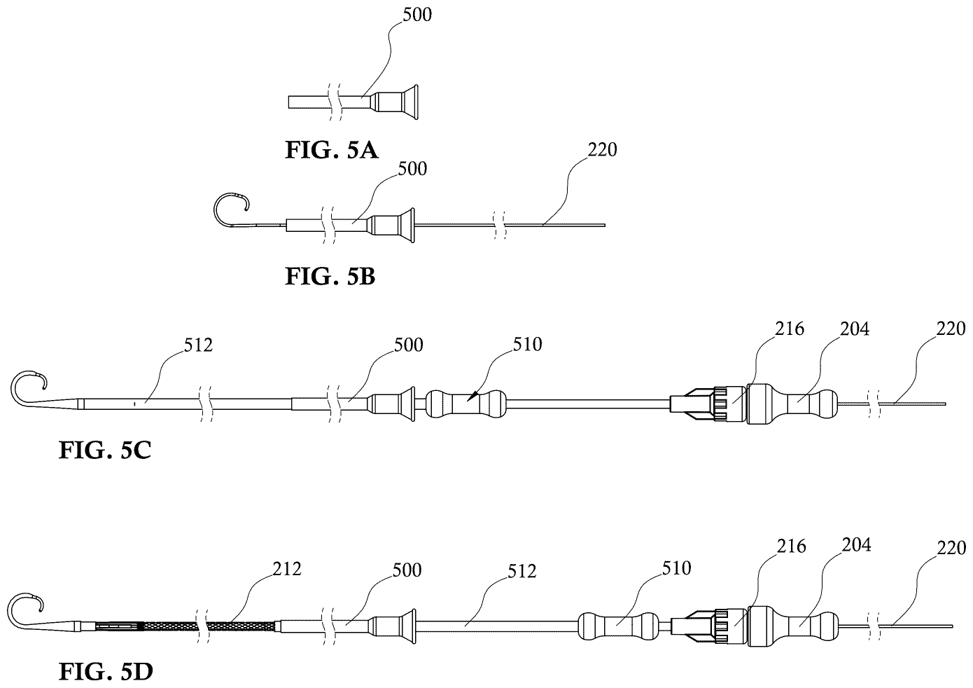

FIGS. 5A-5D illustrate an example sequence for the delivery of the pump housing system when using an independent introducer sheath 500. FIG. 5A illustrates an example introducer sheath 500. FIG. 5B illustrates the introducer sheath 500 with a guidewire 220 placed through the introducer sheath 500. FIG. 5C illustrates tracking the pump housing system over the guidewire 200 and through the introducer sheath 500. The pump housing system is in its collapsed configuration with the delivery sheath 512 encapsulating the expandable cannula and pump housing. In the arrangement depicted in FIG. 5C, the pusher rod shaft is exerting force to tension the inlet cannula and pump housing (e.g., the drive shaft housing hub 216 is coupled to the pusher rod hub 204), thereby maintaining the expandable pump housing and inlet cannula under tension and in a radially collapsed state.

FIG. 5D illustrates the retraction of the delivery sheath 512 by retracting the delivery sheath hub 510 to express and expose the pump housing and inlet cannula 212 in its radially collapsed low-profile delivery state. Having the pusher rod within the pump housing system during expression of the pump housing system also facilitates expression of the expandable pump housing and inlet cannula 212 because it makes the expression forces act as if the collapsed pump housing and inlet cannula 212 are being pulled out of the delivery sheath 512 instead of being pushed out. One advantage of the collapsed pump housing and inlet cannula 212 being pulled out versus pushed out of the delivery sheath 512 is that pulling the system out enhances the tension along the pump housing and inlet cannula 212, biasing it to remain collapsed or even collapse further—whereas if the cannula and pump housing 212 are pushed out, the axial compression along the cannula and pump housing 212 may cause it to start expanding within the delivery sheath 512 (thereby wedging itself within the delivery sheath 512 and increasing the forces required to deliver they cannula and pump housing 212). At this point, full expansion of the pump housing and inlet cannula 212 may be achieved through uncoupling the pusher rod hub 204 from the drive shaft housing hub 216 and the retraction of the pusher rod hub 204. The pusher rod hub 204 may be retracted while leaving the guidewire 220 in place if the clinician desires to maintain wire placement or may be retracted simultaneously with the guidewire 220.

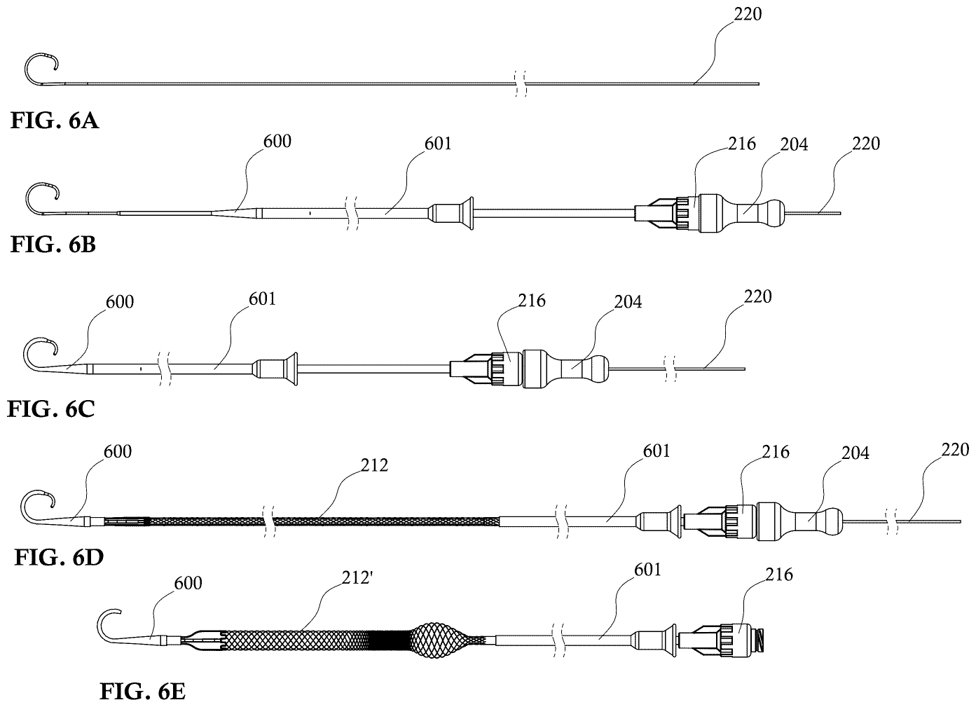

FIGS. 6A-6E illustrate an example sequence for the delivery of the pump housing system when using an integrated introducer sheath 601. FIG. 6A illustrates a guidewire 220 as it would be placed in a patient using standard percutaneous procedures of advancing the guidewire through a hypodermic needle into the patient's vasculature.

FIG. 6B illustrates tracking the pump housing system over the guide wire 220 and into the patient. In this embodiment, the distal tip 600 of the pump housing system also functions as a dilator to expand the size of the vascular access site and allow the rest of the system to track into the patient. Further, there is no dedicated delivery sheath in this embodiment because the dedicated introducer sheath 601 provides the same function by being advanced over the guidewire 220 and into the anatomy simultaneously.

FIG. 6C illustrates the collapsed pump housing system fully inserted over the guidewire.

FIG. 6D illustrates retraction of the dedicated introducer sheath 601 in relation to the pump housing system, thereby expressing and exposing the expandable pump housing and inlet cannula 212. This retraction may be accomplished either by holding the dedicated introducer sheath 601 stationary and further advancing the pump housing system through the dedicated introducer sheath 601, or by holding the pump housing system stationary and withdrawing the dedicated introducer sheath 601 relative to the pump housing system.

FIG. 6E illustrates the fully radially expanded pump housing and inlet cannula 212′ once the pusher rod and guidewire 220 (optionally) are retracted to remove the longitudinal tension from the pump housing and inlet cannula 212. At that point, a separate pump impeller and drive shaft component can be advanced into the fully expanded pump housing and inlet cannula 212′. Then the fully assembled percutaneous ventricular assist device (“pVAD”) can be operated (by rotating the pump impeller and drive shaft) to provide mechanical hemodynamic support to assist with circulating blood throughout the patient's body.

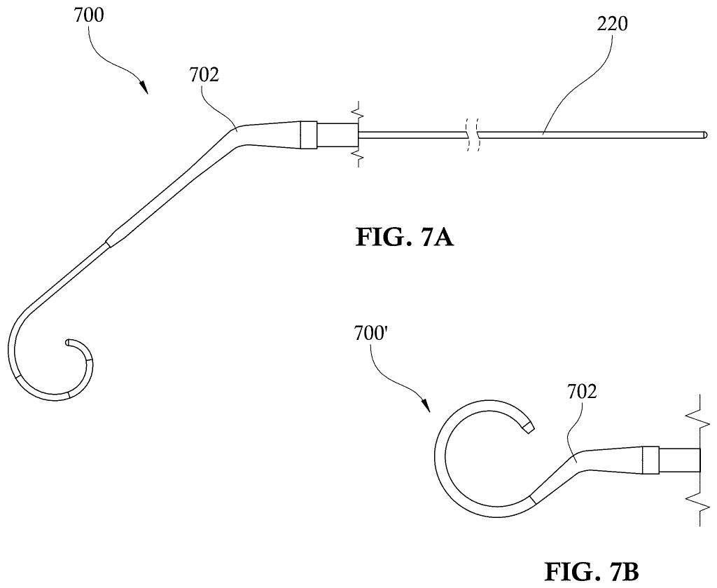

FIGS. 7A and 7B illustrate example embodiments of the distal end member of the pump housing system. FIG. 7A illustrates the distal end member 700 tracked over a guidewire 220. The distal end member 700 has a primary curve 702 which is sufficiently durable to bias the angle of the guidewire 220. This may be advantageous because it may allow for greater steerability of the system within the patient's anatomy.

FIG. 7B illustrates the distal end member 700′ once the guidewire 220 has been removed from the distal end member 700. The very distal end may naturally change its form to be a less traumatic curved tip portion within the patient, however, the primary curve 702 may remain present. Having a primary curve 702 in the distal end member 700′ of the pump housing system, may allow for simplification of the procedure because then the pump housing system may be used to help navigate the guidewire 220 to the desired anatomical location. If the pump housing system cannot provide guidewire steerability, then an auxiliary catheter may be needed to get the guidewire 220 to the needed location which results in more devices being used throughout a procedure and additional procedural steps.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results.

The invention claimed is:

1. A method of deploying a percutaneous ventricular assist blood pump system, the method comprising:
advancing a pump inlet cannula and housing component through a vasculature of a patient to a heart of the patient while the pump inlet cannula and housing component is held in a low-profile delivery configuration resulting from longitudinal tensional force exerted on the pump inlet cannula and housing component by a pusher rod that is abutted against a surface of a distal end member of the pump inlet cannula and housing component;
removing the longitudinal tensional force exerted on the pump inlet cannula and housing component by the pusher rod to allow the pump inlet cannula and housing component to radially expand into an operable configuration;
after the radial expansion of the pump inlet cannula and housing component, inserting a pump impeller and drive shaft component into the pump inlet cannula and housing component while a pump impeller of the pump impeller and drive shaft component is in a low-profiled delivery configuration; and
while the pump impeller is positioned in a pump housing of the pump inlet cannula and housing component, radially expanding the pump impeller to an operable configuration.

2. The method of claim 1, wherein the pump housing is located in an aortic root region of the vasculature when the pump inlet cannula and housing component radially expands into the operable configuration.

3. The method of claim 2, wherein an inlet cannula of the pump inlet cannula and housing component extends across an aortic valve of the heart and into a left ventricle of the heart when the pump inlet cannula and housing component radially expands into the operable configuration.

4. The method of claim 3, further comprising driving rotations of the pump impeller and drive shaft component to pump blood from the left ventricle to the aortic root region via the pump inlet cannula and housing component.

5. The method of claim 1, wherein the pump impeller is radially expanded by supplying an inflation media via a lumen defined by a drive shaft of the pump impeller and drive shaft component.

6. The method of claim 1, wherein a hub of the pusher rod is releasably coupled to a hub of the pump inlet cannula and housing component.

7. The method of claim 6, wherein removing the longitudinal tensional force exerted on the pump inlet cannula and housing component by the pusher rod comprises uncoupling the hub of the pusher rod from the hub of the pump inlet cannula and housing component.

8. The method of claim 1, wherein the pump housing and an inlet cannula of the pump inlet cannula and housing component is contained within an introducer sheath while advancing the pump inlet cannula and housing component to the heart of the patient.

9. The method of claim 8, further comprising, prior to removing the longitudinal tensional force exerted on the pump inlet cannula and housing component by the pusher rod, pulling the introducer sheath proximally to expose the pump housing and the inlet cannula.

10. The method of claim 1, wherein removing the longitudinal tensional force exerted on the pump inlet cannula and housing component by the pusher rod comprises releasing a manual hold on the pusher rod and the pump inlet cannula and housing component.

11. The method of claim 1, wherein, while the longitudinal tensional force is exerted on the pump inlet cannula and housing component by the pusher rod, a shaft of the pusher rod extends through the pump housing and an inlet cannula of the pump inlet cannula and housing component.

12. The method of claim 11, wherein, while the longitudinal tensional force is exerted on the pump inlet cannula and housing component by the pusher rod, a distal tip of the pusher rod is abutted against a distal end member of the inlet cannula.

13. A method of deploying a percutaneous ventricular assist blood pump system, the method comprising:
advancing a pump housing component to a heart of a patient while the pump housing component is in a low-profile delivery configuration resulting from longitudinally tensioning the pump housing component; and
relieving the longitudinal tensioning and to allow the pump housing component to self-expand to an operable configuration.

14. The method of claim 13, further comprising advancing an inner catheter comprising a pump impeller and flexible drive shaft into the pump housing component to assemble the percutaneous ventricular assist blood pump system in vivo.

15. The method of claim 13, wherein the longitudinal tension of the pump housing component is exerted on the pump housing component by a pusher rod.

16. The method of claim 15, wherein, while the longitudinal tension is exerted on the pump housing by the pusher rod, a distal tip of the pusher rod is abutted against a distal end member of an inlet cannula of the pump housing component.

17. The method of claim 16, wherein relieving the longitudinal tensioning and to allow the pump housing component to self-expand to an operable configuration comprises moving the distal tip of the pusher rod away from the distal end member of the inlet cannula.

18. The method of claim 13, wherein advancing the pump housing component to the heart of the patient results in a pump housing of the pump housing component being located in an aortic root region of the vasculature and an inlet cannula of the pump housing component extending across an aortic valve of the heart and into a left ventricle of the heart.
